(19) United States
(12) Patent Application Publication
(10) Pub. No.: US 2007/0202136 A1
(43) Pub. Date: Aug. 30, 2007

(54) Method for reducing the contamination properties of a surface by micro-organisms

(76) Inventors: Gilbert Legeay, Saint-Saturnin (FR); Tanguy Honore, Le Mans (FR); Marie-Noelle Bellon-Fontaine, La Ville Du Bois (FR); Jean-Marie Herry, Egly (FR)

Correspondence Address: YOUNG & THOMPSON, 745 SOUTH 23RD STREET, 2ND FLOOR, ARLINGTON, VA, 22202, US

(21) Appl. No.: 10/591,401

(22) PCT Filed: Mar. 2, 2005
(86) PCT No.: PCT/FR05/50142

§ 371 Date: Nov. 1, 2006

(30) Foreign Application Priority Data
Mar. 3, 2004 (FR) 0450438

(51) Int. Cl. A61K 9/14

(52) U.S. Cl. 424/405; 424/486

(57) ABSTRACT

A method for reducing the contamination properties by micro-organisms of a surface made of a mineral material, the surface having an area of at least 0.1 m², the method including the following steps: a) application, onto the surface to be treated, of a layer of a solution or of an aqueous suspension of a hydrophilic polymeric material; b) drying the surface processed at the step a), for obtaining a surface covered with a layer of the hydrophilic polymeric material.

The present invention relates to the field of the surface treatment against the development of the micro-organisms in environments requiring rigorous aseptic conditions, in particular in the field of agro-foodstuff industries, health industries, in public locations, etc.

The non-controlled development of micro-organisms, in particular bacteriae, fungi or still unicellular algae, but also in some cases viruses, should be avoided as far as possible in sensitive environments such as hospitals, medicament production facilities, the agro-foodstuff industry, the collective kitchens or still public locations.

The contamination of the surfaces by micro-organisms occurs on all the surfaces in contact with the atmosphere, such as soils, technical furniture (operating table, hospital beds, etc), and the production devices used in the pharmaceutical industry and in the agro-foodstuff industry, notably fermentation reactors.

In hospital environment, prevention against contamination of the premises and large furniture by micro-organisms takes on considerable importance when fighting against the propagation of so-called <<nosocomial>> diseases.

In the agro-foodstuff industry, as well as in the field of kitchen for collective facilities, the prevention of contamination by micro-organisms of food production facilities, notably prepared meals, as well as food cooking devices, is essential for causing food intoxications, which are sometimes lethal for the end-user.

Similarly, in industries implementing reactor fermentation methods, it is necessary to avoid the contamination of fermentation media by undesirable exogenous micro-organisms.

It is also important to ensure relative asepsis of numerous locations and objects of public usage, with which numerous individuals are in contact, to prevent germs present on infected individuals from easy dissemination towards sensitive individuals, for example immuno-depressed individuals.

Today, the asepsis of the surfaces is conducted, for example in a hospital environment, by application on these surfaces of solutions containing combinations of antiseptic and bactericidal agents. However, it has been observed that some of the antiseptic and bactericidal agents used are allergenic and exhibit therefore shortcomings regarding human health.

Moreover, some of these agents, although efficient, cannot be used in the agro-foodstuff industry or in culinary premises by reason of their toxicity when ingested by the end consumer

There is hence a need in the state of the art for methods and compositions of surface treatments with a large area, and which may reach in certain cases more than 1000 m², in order to prevent, or at least reduce, the contamination of these surfaces by micro-organisms, and most particularly micro-organisms pathogenic for man or animal.

Taking into account the large area of the surfaces to be treated, it is essential that these anti-biocontamination methods are simple to carry out and little costly.

A method and a composition for preventing or reducing the biocontamination of large surfaces are provided by the present invention.

Surprisingly, the invention shows that a layer of hydrophilic polymer, which prevent the adhesion and the development of the cells of micro-organisms, may be applied with success over a large surface to be treated, of at least 0,1 m², and up to more than 1000 m², in the absence of creation of covalent links between the layer of hydrophilic polymer and the surface treated. It has been shown that the layer of hydrophilic polymer remains in place intact over the surface treated for a duration of several hours, possibly several days, according to the mechanical stresses undergone by said surface treated.

From this surprising result, the applicant has developed a method for reducing the contamination properties by micro-organisms of a surface of a mineral material, for example glass, ceramics, porcelain, cement, concrete or metal, said method including a step during which a layer of a solution of an aqueous suspension of a hydrophilic polymeric material is applied onto the surface to be treated, without creating any covalent links between the layer of hydrophilic polymer and the surface whereon this polymer layer is applied.

The invention relates to a method for reducing the contamination properties by micro-organisms of a surface made of a mineral material, said surface having an area of at least 0.1 m², said method including the following steps:
a) application, onto the surface to be treated, of a layer of a solution or of an aqueous suspension of a hydrophilic polymeric material;
b) drying the surface processed at the step a), for obtaining said surface covered with a layer of said hydrophilic polymeric material.

The layer of the hydrophilic polymeric material applied thanks to the above method adheres to the surface treated by the simple creation of weak non-covalent links between the mineral material of the surface treated and the layer of the polymeric material, in particular weak electrostatic links, hydrogenous links or still Van der Waals forces. The adherence of the layer of the hydrophilic polymeric material on the surface of the mineral material treated does not involve therefore any chemical reaction between said polymeric material and the mineral material. Thus, the surfaces treated by the method of the invention are not modified chemically. These surfaces may hence be treated by the method of the invention repeatedly without being subjected to any modification or alteration.

Nevertheless, surprisingly, the layer of hydrophilic polymeric material adheres stably to the surface of the mineral material. Without wanting to be tied by any given theory, the applicant thinks that the small interactions (hydrogenous links, electrostatic forces, Van der Waals forces) between the surface treated and the layer of hydrophilic polymer suffice in themselves to cause the adherence of said polymeric surface. The adherence properties of the polymeric surface on the surface treated are sufficient so that the polymeric surface remains in position and covers effectively and completely the surface treated, up to the following treatment of this surface by the method of the invention, for example 7, 6, 5, 4, 3, 2 or 1 day(s) after the previous treatment.

Thanks to the method of the invention, the bioadhesion of the micro-organisms on the mineral material is inhibited or blocked, a bioadhesion due to the establishment of electrostatic forces between the bacterial or fungal cells and the mineral material. According to the method of the invention, the bio-adhesion des micro-organisms on a surface while inhibiting or blocking mechanical hooking of these micro-organisms on the surface, often porous or rough, of the mineral material.

Thanks to the method of the invention, the formation de biofilms is also impeded, constituted by a carpet of bacterial or fungal cells which is more or less thick, which are formed rapidly with the course of time, especially in humid atmosphere environments.

Conversely, due to the presence of the layer of the hydrophilic polymeric material, a surface of a mineral material treated according to the above method exhibits small surface energy, which prevents the adhesion of the micro-organisms, notably the bacterial or fungal cells.

Moreover, the external surface, in contact with the outer environment, of the layer of the hydrophilic polymeric material is substantially planar, non porous and non rough, which reduces further the possibility of mechanical hooking of the micro-organisms, notably bacterial or fungal cells, on the surface treated and reduces consequently the biocontamination phenomenon of the surface treated.

By <<mineral material>> is meant according to the invention a material formed of glass, ceramics, porcelain, cement, concrete or still a metallic material.

According to a first preferred embodiment, the mineral material is organic glass or mineral glass.

According to a second preferred embodiment, the mineral material is a metallic material, such as steel, for example a stainless steel, or still aluminium.

As already mentioned previously, the surface of the mineral material treated according to the method of the invention consists of a wide surface having an area of at least 0.1 m². The total area of the surface to be treated is technically only limited by the capacity of the device used for the application of the solution or of the aqueous suspension of the hydrophilic polymeric material. For example, for treating areas above 100 by the method according to the invention, cleaning vehicles may be used, currently employed for cleaning the floors in factories or in public premises.

Thus, surprisingly, the minimum area of the surface which may be treated by the method of the invention is at least 0.1 m² and may be at least 1 m², 2 m², 3 m², 4 m², 5 m², 10 m², 50 m² or at least 100 m². Surfaces up to 1000 m² may be treated.

To implement the method of the invention for the treatment of surfaces having an area smaller than 100 m², one may use suitable devices such as brushes used currently for cleaning small areas.

According to a first aspect, the surface of the mineral material to be treated consists of the surface of a floor, notably the floor of a hospital ward, for example the floor of a hospital bedroom where the patients is being treated, including the floor of an operating room. It may also be the floor of a manufacturing plant of agro-foodstuff products, such as milk agro-foodstuff products (milk, cheese, ice cream, etc.), or still the floor of a kitchen for a collective facility.

The surface of the mineral material to be treated may also consist of the surface of devices or furniture used in hospitals or in the industry, such as the surface of the tray of a hospital table, including an operating table, or still a culinary work top.

The surface of the mineral material to be treated may also consist of the surface of tubings or fluid circulation pipes, notably in hospitals and in the agro-foodstuff industry, including the tubings or pipes for circulating gases or liquid fluids such as water.

According to another aspect still, the surface of the mineral material to be treated may consist of the internal and/or external surface of a fermentation reactor used for the fabrication of various metabolites, for example in the agro-foodstuff industry or still in the pharmaceutical industry.

Preferably, in step a) of the method, the solution or the aqueous suspension includes the hydrophilic polymeric material at a concentration ranging between 0.5% and 5%, better between 1% and 3%, based on the total weight of the solution or of the aqueous suspension.

The higher the concentration of the hydrophilic polymeric material of aqueous solution, the greater the viscosity of the aqueous solution and the greater and more protective the thickness of the layer of the hydrophilic polymeric material on the surface treated. According to the mechanical stresses which may be inflicted to the surface to be treated, such as for example the frequent displacement of objects on castors, the traffic of numerous individuals, etc., the concentration of the hydrophilic polymeric material is adjusted so that the layer of the hydrophilic polymeric material has sufficient thickness to prevent the development of the micro-organisms up to the following treatment of said surface by the method.

In order to obtain sufficiently protective a layer of hydrophilic polymer, i.e. a layer of hydrophilic polymer of sufficiently thickness for covering without excessive difficulty the whole surface to be treated, the quantity of the hydrophilic polymeric material in relation to the total weight of the solution is obtained so as to provide an aqueous solution of the hydrophilic polymeric material having a viscosity of at least 1 centipoise.

The viscosity of the aqueous solution or aqueous suspension of the hydrophilic polymeric material may reach 10 centipoises, when a layer of hydrophilic polymeric material of great thickness is applied, for example on surfaces of mineral materials intended for being subjected to numerous mechanical stresses.

According to the invention, the viscosity measurements are realised in a Brookfield system according to a standardised technique, at controlled temperature (25° C.), by using a DIN viscosimeter fitted with a DIN 30D-type needle, and for a rotational speed of 300 to 500 revolutions/minute. The piece of equipment gives automatically the viscosity value which is a function of a rotational torque.

Quite preferably, the hydrophilic polymeric material consists of a polymer or a combination of polymers selected among the celluloses and their derivatives, the polyacrylamides and their copolymers, the polyvinyl pyrrolidone (PVP) and its copolymers, the vinyl acetate copolymers and vinyl alcohol copolymers, the glycol polyethylene, the polypropylene glycols, the hydrophilic polyacrylates, the hydrophilic polymethacrylates, the polyosides and the chitosans.

Preferably, the hydrophilic polymeric material is selected among the following hydrophilic polymers:
the celluloses and their derivatives, such as the hydroxypropyle methylcellulose (HPMC), for example the HPMC E4M marketed by DOW CHEMICALS, or that designated as Aqualon marketed by Herculés, or still the carboxy methylcellulose (CMC) marketed by DOW CHEMICALS:
the polyacrylamides and their copolymers, such as those marketed by SIGMA (UPSALA, Sweden);
the polyvinyl pyrrolidone (PVP) and its copolymers, such as those marketed by BASF/LASERSON, such as the family of Kollidons;
the copolymers of vinyl acetate, such as the vinyl polyacetate copolymers and polyvinyl alcohol copolymers marketed under the name of Mowiol by HOECHST/CLARIANT.

the glycol polyethylenes, such as those marketed by SIGMA.

the glycol polypropylenes;
the poly hydrophilic (meth)acrylates, such as those marketed by DEGALAN or DEGUSSA;
the polyosides;
the chitosans, such as those marketed by SIGMA.

By hydrophilic polymeric material according to the invention is meant a polymeric material formed of one of the hydrophilic polymers such as defined above, as well as a mixture of several of the above hydrophilic polymers, generally, a mixture of two or three of the above hydrophilic polymers.

It has been shown according to the invention that surfaces of glass or of steel covered with a layer of hydrophilic polymeric materials with the method of the invention was stable on the surface treated for a duration up to 96 hours.

It has also been shown that a surface of glass or of treated steel, according to the method of the invention, by a layer of a hydrophilic polymeric material, after immersion during 72 hours in a solution containing bacterial cells, such as Escherichia coli and Staphyloccus epidermis at concentrations ranging between 10⁶ and 3.10⁶ per gram of solution, had not been colonised by these bacteriae. Significant mortality has been observed with the cells of Escherichia coli, with a diminution by a factor 2 relative to the number of cells inoculated initially. For the Staphylococus epidermis, the mortality after 72 hours is almost complete. Notably, no film of bacterial cells has been observed at the surface of the material treated. In comparison, the same medium, non treated according to the method of the invention, possesses, at the end of 72 hours immersion in the same solution containing bacteriae, a coverage ratio by the bacteriae of the order of 70% of its total area.

With a surface treated according to the method of the invention, a slide of the bacterial cells over the layer of hydrophilic polymer has been observed, without fixation of these cells to the surface of said layer of hydrophilic polymer.

At step a) of the method according to the invention, the application of the hydrophilic polymeric material is conducted, according to the type and the geometry of the device to be treated, either by quenching the surface to be treated, or by application of the material en hydrophilic polymer onto the surface to be treated using a brush, a pencil, or of any type of spaying device, such as paint spraying gun or still a high-pressure cleaning device, such as a Karcher® type device.

One may also use a cloth surface, for example a wiping cloth, previously impregnated with the solution or suspension of the hydrophilic polymeric material.

At step b) of the method, the drying may be realised simply without any human intervention, for sufficient duration necessary to the evaporation of the aqueous solvent in contact with the surrounding atmosphere.

The drying step b) may be also realised by the application, on the surface treated according to step a), of an air stream at room temperature of approx. 20 to 25° C., or still of an air stream at a temperature which may reach 55° C., for sufficient duration to cause the evaporation of the aqueous solvent, for example approx. 10 minutes, on each potion of the surface treated.

The invention also concerns a composition for the treatment of a surface of a mineral material having an area of at least 0.1 m² against contamination by micro-organisms, characterised in that said composition consists of an aqueous solution of a hydrophilic polymeric material including at least one bactericidal agent or a preservative agent.

According to the viscosity of the solution or of the aqueous suspension of the hydrophilic polymeric material, it has been observed that the method of the invention enabled to apply onto the surface to be treated a layer of hydrophilic polymer ranging from approx. 100 nanometres to 10 μm.

By way of example, a value of viscosity of the solution or suspension of the hydrophilic polymeric material of the order of 5 to 10 centipoises (cPs) is obtained for a concentration of 1% in weight of PVP (Kollidon K90 marketed by BASF) or for a concentration of 0.2% in weight of HPMC (E4M marketed by DOW CHEMICALS).

By <<aqueous solution or aqueous suspension>> according to the invention is meant mainly that the hydrophilic polymeric material used is a solution or a suspension in water, possibly in the presence of a small proportion, for example from 0.1% to 5% (V:V), of a water-miscible solvent such as ethanol or a ketone, including acetone, or any other diluent authorised by administrative regulations.

The aqueous solution containing the hydrophilic polymeric material may also contain one or several additives intended for improving the spreading thereof onto the surface to be treated such as neutral surfactant agents or with an acid or basic character. Neutral surfactant additives are preferred however, such as polyethylenes and glycol polypropylenes, stearate polyoxyethyl sorbitol (Tween®), a polyether or still a polysiloxane-polyether. These products are notably marketed by BYK CHEMIE.

The aqueous solution of the hydrophilic polymeric material may also include an adhesion promoting agent for better adherence of the polymer layer on the surface treated, such as organo-silicated, phosphor-aluminates or zircon-aluminates, or phosphates. The latter are most favoured. All of them are of commercial origin. In certain cases, the acid or basic character should be taken into account relative to the chemical nature of the medium whereon the coating is applied, in order to prevent any alteration of the surface to be treated.

Advantageously, the aqueous solution of the hydrophilic polymeric material contains one or several antiseptic agents, one or several antibacterial agents or one or several preservative agents, which enables further reduction of the proliferation of bacterial or fungal cells. One may use for example, as a preservative agent, sodium benzoate, preferably at the final weight concentration of 1%, or still sodium azide, preferably at the final weight concentration of 1%.

In order to treat, thanks to the method of the invention, surfaces intended for exposure to high or numerous mechanical stresses, it may be in certain cases advantageous to add to the aqueous solution of the hydrophilic polymeric material a hardening agent capable of improving the resistance of the layer to abrasion.

Quite preferably, the hardening agent consists of synthetic silica particles dispersed in aqueous liquid medium. The synthetic silica particles are formed of silicic acid polymers whereof the mass structure is produced by reticulation of the SiO₄ tetrahedra. At the surface of the particles, the structure of the polymers is terminated by siloxane groups and silanols groups. Particles of hydrophilic fumed silica are used preferably. The fumed silicae are prepared by hydrolysing silicon tetrachloride (TiCl₄) in vapour phase in a hydrogen and oxygen flame at a temperature of at least 1000° C. having a specific surface area ranging between 200 and 400 m² μg, better still between 250 and 400 m²/g, and most preferably between 300 and 400 m²/g, for example silicae having a specific surface area of 315 m²/g or of 342 m²/g. One may use notably silica dispersions marketed under the name Aérosil by DEGUSSA. Among the Aérosil-type silica dispersions, those having the grades K315 and K 342, respectively, are used preferably.

Most preferably, silica particles dispersed in an aqueous liquid medium with slightly acid character, such as the silicae K315 and K342 above are used

Conversely, dispersions of highly basic silica particles are avoided, because of the risk of dissolution of this silica in the medium to form silicates, which is not desirable.

Preferably, according to this particular aspect of the method, the aqueous solution or aqueous dispersion of the polymeric material contains from 50 g/l to 250 g/l of silica particles, and most preferably from 70 g/l to 200 g/l of silica particles.

It has also been shown according to the invention that the layer of hydrophilic polymer obtained by application of the method with an aqueous solution of 0.2% E4M-type HPMC in weight in water had a 2.33 N.mm⁻² hardness, as determined by micro indentation (Fisher H100C system). A pyramid-shape indenter (136°) driven by a microscope penetrates into the material under a 25 mN load within 20 sec (temperature 22° C. and humidity ratio 45%) so that the indenter does not penetrate over more than a quarter of the thickness of the layer studied. Measuring the depth of penetration reached at each maximum load enables to calculate the hardness.

It has also been shown according to the invention that the hardness of the polymeric surface obtained by application of the method with an aqueous solution of 0.2% E4M-type HPMC in water containing 50% in weight of a dispersion of K315-type silica particles had a hardness of approx. 350 N.mm⁻², as determined by micro indentation.

The composition according to the invention includes preferably from 0.5% to 5% in weight, in relation to the total weight of the aqueous solution, of said hydrophilic polymeric material.

The invention also relates to a surface of a mineral material having an area of at least 1 m² and resistant to contaminations by micro-organisms, characterised in that said glass, ceramics or steel surface is covered with a layer of a hydrophilic polymeric material.

The above surface is characterised in that the layer of hydrophilic polymeric material includes the silica particles and has a surface hardness greater than 100 N.mm⁻². The results with Eschericcia coli and Staphylococcus epidermis show an absence of adhesion after 24 hours

The invention is also illustrated by the following examples.

### EXAMPLES

### Example 1

### Adherence and Biocontamination Test on a Glass Surface Treated with a Hydrophilic Polymer According to the Method of the Invention

A. Protocol for Preparing Glass Plates Covered with a Hydrophilic Polymer.

The glass plates are washed (i) either with soapy water than rinsed in pure water, (ii) or with a clean organic solvent (and notably ethyl alcohol). In all cases, the plates are dried in room air if possible under a hot air stream, for a few minutes. These plates are kept in a clean environment.

Then, the surface of the plates is covered with a layer of hydrophilic polymer by immersion in a bath of a 0.2% HPMC solution (w/w) for a duration of 72 hours

B. Results of the Bioadherence tests.

Bioadherence tests are conducted in physiological medium:
either in a static system, i.e. the objects are soaked for several days and samples are taken regularly,
or in a dynamic system, by immersing the surface treated according to the method of the invention into a tub containing a solution of a medium containing micro-organisms. Currents of the liquid medium are generated inside the tub, in order (i) to test the adhesion capacity of the layer of hydrophilic polymer on the surface of the medium and (ii) to test the anti-biocontamination capacity of the surface treated. Solutions with a concentration of the order of 1.10⁶ micro-organisms cm⁻³ are used.
   Results Obtained by Application of the Method of the Invention onto a Glass Surface:

Glass platelets covered with a hydrophilic polymer (HPMC or hydroxypropyl methylcellulose) are brought in contact for durations up to 72 hours. In certain cases, the plates are washed with a phosphated alkali detergent (RBS).

The results are represented on Table 1 below.

TABLE 1

| | | | AFTER TEST |
|---|---|---|---|
| | | | % surface covered |
| | BEFORE TEST | | by the micro- |
| Surface | Treated HPMC | + preparation | organisms |
| Glass | no | RBS washing | 70% |
| | yes | no washing | 0% |
| | yes | RBS washing | 0% < S < 10% |
| Steel | no | No washing | 100% |
| | yes | RBS washing | 10% |

### Example 2

### Adherence and Biocontamination Test on a Glass Surface Treated with an Association of a Hydrophilic Polymer with Silica Particles According to the Method of the Invention

A. Protocol for Preparing Glass Plates Covered with a Layer Containing a Mineral Load of Silica Particles

Preparation of the Glass Plates: they are Washed Either with Soapy water than rinsed in pure water, or with a clean organic solvent (and notably ethyl alcohol). In all cases, the plates are dried in room air if possible under a hot air stream, for a few minutes. These plates are kept in a clean environment.

Then, the surface of the plates is covered with a layer of hydrophilic polymer:
either by immersion in a bath of a 0.2% HPMC solution (w/w) for a duration of 72 hours
or by immersion into a bath of 100 ml 0.2% HPMC solution (w/w) to which 1.33 ml of an aqueous solution of 15.0 g/l K315 silica particles have been added.
   B. Results of the Bioadherence Tests.

Glass platelets covered with a hydrophilic polymer (HPMC or hydroxypropyl methylcellulose) and containing a mineral load of silica particles are brought in contact for durations up 72 hours. In certain cases the plaques are washed with a phosphated alkali detergent (RBS). The composition of the hydrophilic polymer solution is 100 ml of an aqueous solution of 0.2% HPMC (w/w) to which 1.33 ml of an aqueous solution of 150 g.l⁻¹ K315 silica particles have been added, which correspond, for the dry extract, to 50 parts in weight of polymer and 50 parts in weight of silica particles.

The results are represented on Table 2 below.

TABLE 2

| | | HPMC deposit + Silica + | HPMC deposit + Silica + |
|---|---|---|---|
| Strain | Without any deposit | rinsing with distilled water | 2% RBS35 cleaning |
| E. coli | Adherence of the micro- | No adherence of the micro- | The spots that could be seen |
| | organisms after de 24 | organisms after 24 h | previously have disappeared |
| | hours | | (surface more uniform) |
| | % surface covered with the | % surface covered with the | Little adherence of the micro- |
| | microorganisms: 70% | microorganisms: 0% | organisms after 24 h |
| | | | % surface covered with the |
| | | | microorganisms: 2% |
| S. | | Before test: very opaque | Before test: the spots that could be |
| epidermis | | surface (tuning difficult to be | seen previously have disappeared |
| | | conducted) | (surface more uniform) |
| | | At first, no adherence of the | Strong adherence of the micro- |
| | | microorganisms after 24 hours, | organisms after 24 h |
| | | but the surface is decaying in | |
| | | little pieces. | |
| | | % surface covered with the | % surface covered with the |
| | | microorganisms: 0% | microorganisms: 50% |

1. A method for reducing the contamination properties by micro-organisms of a surface made of a mineral material, said surface having an area of at least 0,1 m², said method including the following steps:
a) application, onto the surface to be treated, of a layer of a solution or of an aqueous suspension of a hydrophilic polymeric material;
b) drying the surface processed at the step a), for obtaining said surface covered with a layer of said hydrophilic polymeric material.

2. A method according to claim 1, characterised in that the mineral material is organic glass or mineral glass.

3. A method according to claim 1, characterised in that the mineral material is selected among ceramics, porcelain, cement or concrete.

4. A method according to claim 1, characterised in that the mineral material is a metallic material, such as steel or aluminium.

5. A method according to claim 1, characterised in that the surface of mineral material consists in the surface of a soil, of a culinary work top, of a table, of a bed, of a reactor, or still of a tubing.

6. A method according to claim 1, characterised in that, in step a), the solution or the aqueous suspension includes the hydrophilic polymeric material at a concentration ranging between 0.5% and 5% in weight, preferably between 1% and 3% in weight, based on the total weight of the solution or of the aqueous suspension.

7. A method according to claim 6, characterised in that the hydrophilic polymeric material is selected among the celluloses and their derivatives, the polyacrylamides and their copolymers, the polyvinyl pyrrolidone (PVP) and its copolymers, the vinyl acetate copolymers and vinyl alcohol copolymers, the glycol polyethylenes, the glycol polypropylenes, the hydrophilic polyacrylates, the hydrophilic polymethacrylates, the polyosides and the chitosans.

8. A method according to any of the claim 1, characterised in that the solution or suspension of hydrophilic polymeric material includes moreover silica particles dispersed in the aqueous medium.

9. A method according to claim 8, characterised in that the solution or suspension of hydrophilic polymeric material has a content of silica particles ranging from 50 g.l⁻¹ to 250 g.l⁻¹.

10. Method according to claim 1, characterised in that in step a), the application of the hydrophilic polymeric material is conducted by quenching the surface to be treated, or by application of the material onto the surface using a brush, a roll or a spraying device.

11. A composition for the treatment of a surface having an area of at least 1 m² against contamination by micro-organisms, characterised in that said composition consists of an aqueous solution of a hydrophilic polymeric material, said composition including silica particles with a final content ranging from 50 g.l⁻¹ to 250 g.l⁻¹, and including at least one bactericidal agent or a preservative agent.

12. A surface of a mineral material having an area of at least 1 m² and resistant to contaminations by micro-organisms, characterised in that said glass, ceramics or steel surface is covered with a layer of a hydrophilic polymeric material.

13. A surface according to claim 11, characterised in that the layer of hydrophilic polymeric material includes silica particles and has a surface hardness above 100 N.mm⁻².
